(19) United States
(12) Patent Application Publication
(10) Pub. No.: US 2026/0265448 A1
(43) Pub. Date: Sep. 10, 2026

(54) POLYMER COMPOSITIONS WITH THERMALLY CONTRACTILE MOLECULES

(71) Applicant: National Technology & Engineering Solutions of Sandia, LLC, Albuquerque, NM (US)

(72) Inventors: Erica Marie Redline, Albuquerque, NM (US); Eric Michael Nagel, Albuquerque, NM (US); Chad Staiger, Albuquerque, NM (US)

(21) Appl. No.: 19/075,246

(22) Filed: Mar. 10, 2025

(51) Int. Cl. C08G 59/50; C07D 321/12; C08G 69/40

(52) U.S. Cl. CPC C08G 59/5046; C07D 321/12; C08G 69/40

(57) ABSTRACT

A polymer composition includes a polymeric moiety covalently attached to a bisdibenzo-1,4-dioxocane having a coefficient of thermal expansion that is less than a coefficient of thermal expansion of the polymeric moiety. Also provided are methods for synthesizing a substituted bisdibenzo-1,4-dioxocane and for preparing a polymer composition.

### STATEMENT OF GOVERNMENTAL INTEREST

This invention was made with Government support under Contract No. DE-NA0003525 awarded by the United States Department of Energy/National Nuclear Security Administration. The U.S. Government has certain rights in the invention.

### FIELD OF INVENTION

The present invention relates to polymer compositions and, in particular, to polymer compositions comprising polymeric moieties covalently bound to thermally contractile molecules and related methods.

### BACKGROUND

Polymers are low weight, low-cost, high-performance materials with excellent chemical, thermal, and mechanical stability. In addition to their use in homogenous components, polymers are frequently employed in combination with other materials, acting as adhesives, encapsulants, composite matrices, or barriers. In these cases, additional practical constraints are imposed upon the resulting materials, including the need to closely match the thermal expansion behaviors of the various constituents to achieve optimal performance.

Most solid materials experience positive thermal expansion upon heating, and the degree and rate at which this expansion occurs is referred to as the coefficient of thermal expansion (CTE). Bulk polymers typically possess large, positive CTEs in comparison to other materials, such as ceramics or metals. For example, a representative CTE value of a cured epoxy is approximately 55 ppm/° C., whereas common inorganic fillers such as silica or alumina possess CTE values of approximately 6 ppm/° C. and approximately 8 ppm/° C., respectively. When combined, the fillers can reduce CTE, but also impart added weight and stiffness. In composites or devices, large differences in CTE between materials is known as CTE mismatch, which can cause internal thermomechanical stresses that may reduce reliability, the service life of the component and, in some cases, catastrophic device failure. Fluoroelastomers and rubbers used in high temperature applications, such as seals for geothermal, oil, and gas applications, can also suffer from CTE issues. As such, fine-tuning of polymer CTE represents a significant scientific challenge of interest to a variety of industries.

One strategy to address CTE mismatch is to incorporate negative thermal expansion (NTE) materials as fillers within the polymer matrix. Such fillers act to depress the overall CTE of the composite. Inorganic compounds such as ZrW208 (CTE of approximately −9 ppm/° C.), or GaNMn₃ (CTEs as low as −70 ppm/° C.), have been explored for this purpose, allowing for the CTE of their respective composites to be modulated over an order of magnitude depending on filler loading. However, despite their promise, such composite materials are typically limited in their useful CTE window to sub-ambient temperatures. Moreover, high loadings of inorganic fillers are often required (80-90 wt. %) to significantly reduce CTE values, which can hinder material processing, add weight, and dramatically alter morphology and mechanical performance.

Another strategy is to crosslink a low or negative CTE molecule into the backbone of the polymers in the matrix. However, many of these low/negative CTE molecules have very complex synthetic routes with low yields, which may substantially increase the time and expense of manufacturing and hinder commercialization efforts.

Accordingly, there is a need for covalently bound polymer compositions with low/negative CTE molecules, wherein the low/negative CTE molecules have simple and scalable synthetic routes, while the molecules are still effective at lowering the CTE of the polymer composition.

### SUMMARY

The following is a brief summary of subject matter that is described in greater detail herein. This summary is not intended to be limiting as to the scope of the claims.

According to a first aspect of the present disclosure, a polymer composition comprises a polymeric moiety covalently bound to a bisdibenzo-1,4-dioxocane having the below structure:

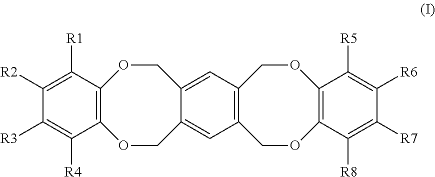

wherein at least one of R1-R8 comprises at least one covalent attachment site for the polymeric moiety; wherein the polymeric moiety is a polymer or a polymer resin; and wherein the polymer composition has a coefficient of thermal expansion (CTE) that is less than a coefficient of thermal expansion of the polymer or polymer resin.

According to a second aspect of the present disclosure, a polymer composition comprises the polymer composition of the first aspect, wherein the polymeric moiety is a polymer and the polymer composition is a thermoplastic polymer composition.

According to a third aspect of the present disclosure, a polymer composition comprises the polymer composition of the first aspect, wherein the polymeric moiety is a polymer resin and the polymer composition comprises a thermoset polymer crosslinked to the bisdibenzo-1,4-dioxocane.

According to a fourth aspect of the present disclosure, a polymer composition comprises the polymer composition of the first or second aspect, wherein the polymeric moiety is selected from the group consisting of a polystyrene, a polyolefin, a polyurethane, a polyester, a polyamide, a polyimide, and a polyepoxide.

According to a fifth aspect of the present disclosure a polymer composition comprises the polymer composition of any of the previous aspects, wherein the bisdibenzo-1,4-dioxocane comprises two or more covalent attachment sites, with one or more covalent attachment sites at one or more of R1-R4 and one or more covalent attachment sites at one or more of R5-R8.

According to a sixth aspect of the present disclosure, a polymer composition comprises the polymer composition of any of the previous aspects, wherein the bisdibenzo-1,4-dioxocane is a disubstituted-bisdibenzo-1,4-dioxocane having two covalent attachment sites, with one covalent attachment site at one of R1-R4 and another covalent attachment site at one of R5-R8.

According to a seventh aspect of the present disclosure, a polymer composition comprises the polymer composition of the sixth aspect, wherein the disubstituted-bisdibenzo-1,4-dioxocane comprises a cis isomer.

According to an eighth aspect of the present disclosure, a polymer composition comprises the polymer composition of the sixth aspect, wherein the disubstituted-bisdibenzo-1,4-dioxocane comprises a trans isomer.

According to a ninth aspect of the present disclosure, a polymer composition comprises the polymer composition of any of the previous aspects, wherein the at least one covalent attachment site comprises a first covalent bond between a functional group substituent and the bisdibenzo-1,4-dioxocane, and a second covalent bond between the functional group substituent and the polymeric moiety.

According to an tenth aspect of the present disclosure, a method for synthesizing a substituted bisdibenzo-1,4-dioxocane comprises: (1) producing a reaction mixture comprising a bisdibenzo-1,4-dioxocane intermediate having at least one reactive moiety on at least one of its terminal benzene groups; (2) adding a functional group reactant to the reaction mixture; and (3) converting the at least one reactive moiety on the bisdibenzo-1,4-dioxocane intermediate to at least one functional group substituent to produce a substituted bisdibenzo-1,4-dioxocane having the below structure:

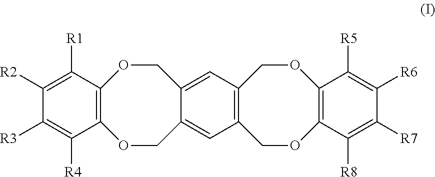

wherein at least one of R1-R8 comprises the at least one functional group substituent.

According to an eleventh aspect of the present disclosure, a method for synthesizing a substituted bisdibenzo-1,4-dioxocane comprises the method according to the tenth aspect, wherein the substituted bisdibenzo-1,4-dioxocane is a disubstituted bisdibenzo-1,4-dioxocane with two functional group substituents, wherein one of R1-R4 comprises the first functional group substituent, and one of R5-R8 comprises the second functional group substituent.

According to a twelfth aspect of the present disclosure, a method for synthesizing a substituted bisdibenzo-1,4-dioxocane comprises the method according to the tenth or eleventh aspects, wherein there is more than one functional group substituent, and wherein all of the functional group substituents are the same.

According to a thirteenth aspect of the present disclosure, a method for synthesizing a substituted bisdibenzo-1,4-dioxocane comprises the method according to any of the tenth to twelfth aspects, wherein the at least one functional group substituent is selected from the group consisting of: an amine, an epoxide, an azido, a hydroxy, an isocyanate, a carboxylic acid, a vinyl, an acrylate, a methacrylate, and an anhydride group.

According to a fourteenth aspect of the present disclosure, a method for synthesizing a substituted bisdibenzo-1,4-dioxocane comprises the method according to any of the tenth to thirteenth aspects, wherein the functional group reactant is selected from the group consisting of: a metal-bis(trimethylsilyl)amide, an organic peroxide, a sodium azide, a bis(trimethylsilyl) peroxide, a phosgene, a bis(trichloromethyl) carbonate, a carbon dioxide, a tributylvinyltin, a methyl acrylate, a methyl methacrylate, and an acetyl chloride.

According to a fifteenth aspect of the present disclosure, a method for synthesizing a substituted bisdibenzo-1,4-dioxocane comprises the method according to any of the tenth to fourteenth aspects, wherein the at least one reactive moiety on the bisdibenzo-1,4-dioxocane intermediate is selected from the group consisting of: a halide, a vinyl, a diamine, a hydroxyl, and a carboxylic acid.

According to a sixteenth aspect of the present disclosure, a method for synthesizing a substituted bisdibenzo-1,4-dioxocane comprises the method according to the fifteenth aspect, wherein the halide is selected from the group consisting of: bromine, iodine, and chlorine.

According to a seventeenth aspect of the present disclosure, a method for synthesizing a substituted bisdibenzo-1,4-dioxocane comprises the method according to any of the tenth to sixteenth aspects, wherein the addition step further comprises adding a catalyst and/or a stoichiometric reagent to the reaction mixture.

According to an eighteenth aspect of the present disclosure, a method for synthesizing a substituted bisdibenzo-1,4-dioxocane comprises the method according to the seventeenth aspect, wherein the catalyst is selected from the group consisting of: a transition metal, a tri-tert-butylphosphine, a sodium nitrite, and a sodium-tert-butoxide.

According to a nineteenth aspect of the present disclosure, a method for synthesizing a substituted bisdibenzo-1,4-dioxocane comprises the method according to the eighteenth aspect, wherein the catalyst is a transition metal, and wherein the transition metal is selected from the group consisting of: palladium, platinum, rhodium, ruthenium, and iridium.

According to a twentieth aspect of the present disclosure, a method for synthesizing a substituted bisdibenzo-1,4-dioxocane comprises the method according to any of the seventeenth to nineteenth aspects, wherein: the step of producing the reaction mixture comprises mixing a 4-halocatechol and a 1,2,4,5-tetra(halomethyl)benzene with a potassium carbonate, an acetonitrile, and a dimethylformamide; the at least one reactive moiety comprises two halides and the bisdibenzo-1,4-dioxocane intermediate comprises a dihalide-bisdibenzo-1,4-dioxocane having one halide on each of its terminal benzene groups; the functional group reactant comprises a metal-bis(trimethylsilyl)amide; the catalyst comprises a transitional metal and a tri-tert-butylphosphine; and the at least one functional group substituent comprises two amines, wherein one of R1-R4 comprises the first amine, and one of R5-R8 comprises the second amine.

According to a twenty-first aspect of the present disclosure, a method for synthesizing a substituted bisdibenzo-1,4-dioxocane comprises the method according to the twentieth aspect, wherein the metal in the metal-bis(trimethylsilyl)amide is selected from the group consisting of: lithium, sodium, and potassium.

According to a twenty-second aspect of the present disclosure, a method for synthesizing a substituted bisdibenzo-1,4-dioxocane comprises the method according to the twentieth or twenty-first aspect, wherein the method further comprises adding a dilute aqueous acid in a miscible organic solvent to the reaction mixture following the addition of the functional group reactant and the catalyst.

According to a twenty-third aspect of the present disclosure, a method for synthesizing a substituted bisdibenzo-1,4-dioxocane comprises the method according to the twenty-second aspect, wherein the dilute aqueous acid is hydrochloric acid.

According to a twenty-fourth aspect of the present disclosure, a method for synthesizing a substituted bisdibenzo-1,4-dioxocane comprises the method according to the twenty-second aspect, wherein the miscible organic solvent is tetrahydrofuran.

According to a twenty-fifth aspect of the present disclosure, a method for synthesizing a substituted bisdibenzo-1,4-dioxocane comprises the method according to any of the twentieth to twenty-fourth aspects, wherein the transition metal is palladium.

According to a twenty-sixth aspect of the present disclosure, a method of preparing a polymer composition comprises: (1) mixing a polymeric moiety with a substituted-bisdibenzo-1,4-dioxocane having the below structure:

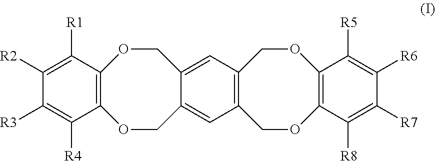

wherein at least one of R1-R8 comprises at least one functional group substituent and wherein the polymeric moiety is selected from the group consisting of: a monomer, a polymer, and a polymer resin; and (2) covalently bonding the at least one functional group substituent of the substituted-bisdibenzo-1,4-dioxocane to the polymeric moiety.

According to a twenty-seventh aspect of the present disclosure, a method of preparing a polymer composition comprises the method according to the twenty-sixth aspect, wherein the substituted bisdibenzo-1,4-dioxocane is a disubstituted bisdibenzo-1,4-dioxocane with two functional group substituents, wherein one of R1-R4 comprises the first functional group substituent, and one of R5-R8 comprises the second functional group substituent.

According to a twenty-eighth aspect of the present disclosure, a method of preparing a polymer composition comprises the method according to the twenty-sixth or twenty-seventh aspect, wherein there is more than one functional group substituent, and wherein all of the functional group substituents are the same.

According to a twenty-ninth aspect of the present disclosure, a method of preparing a polymer composition comprises the method according to any of the twenty-sixth to twenty-eighth aspects, wherein the at least one functional group substituent is selected from the group consisting of: an amine, an epoxide, an azide, a hydroxy, an isocyanate, a carboxylic acid, a vinyl, an acrylate, a methacrylate, and an anhydride group.

According to a thirtieth aspect of the present disclosure, a method of preparing a polymer composition comprises the method according to any of the twenty-sixth to twenty-ninth aspects, wherein the mixing step further comprises mixing a linking group with the polymeric moiety and the substituted bisdibenzo-1,4-dioxocane, and wherein the covalent bonding step comprises the linking group forming a covalent bond with the polymeric moiety and the linking group forming a covalent bond with the at least one functional group substituent.

According to a thirty-first aspect of the present disclosure, a method of preparing a polymer composition comprises the method according to any of the twenty-sixth to twenty-ninth aspects, wherein the covalent bonding step comprises the polymeric moiety forming a covalent bond with the substituted-bisdibenzo-1,4-dioxocane.

According to a thirty-second aspect of the present disclosure, a method of preparing a polymer composition comprises the method according to any of the twenty-sixth to thirty-first aspects, wherein the polymeric moiety is a polymer or polymer resin, and wherein the polymeric moiety is selected from the group consisting of a polystyrene, polyolefin, a polyurethane, a polyester, a polyamide, a polyimide, and an epoxy.

According to a thirty-third aspect of the present disclosure, a method of preparing a polymer composition comprises the method according to the thirty-second aspect, wherein the polymer composition comprises the substituted bisdibenzo-1,4-dioxocane covalently bound to the polymer or polymer resin.

According to a thirty-fourth aspect of the present disclosure, a method of preparing a polymer composition comprises the method according to any of the twenty-sixth to thirty-first aspects, wherein the polymeric moiety is a monomer, and wherein the method further comprises a polymerization of the monomer following the covalent bonding step.

According to a thirty-fifth aspect of the present disclosure, a method of preparing a polymer composition comprises the method according to the thirty-fourth aspect, wherein the monomer is selected from the group consisting of: a styrene, an olefin, a carbamate, an ester, an amide, an imide, methacrylate, acrylate, vinyl, and an epoxy.

According to a thirty-sixth aspect of the present disclosure, a method of preparing a polymer composition comprises the method according to the thirty-fourth or thirty-fifth aspect, wherein the polymer composition comprises the substituted bisdibenzo-1,4-dioxocane covalently bound to the polymerized monomer.

The above summary presents a simplified summary in order to provide a basic understanding of some aspects of the compositions and/or methods discussed herein. This summary is not an extensive overview of the compositions and/or methods discussed herein. It is not intended to identify key/critical elements or to delineate the scope of such compositions and/or methods. Its sole purpose is to present some concepts in a simplified form as a prelude to the more detailed description that is presented later.

### BRIEF DESCRIPTION OF THE DRAWINGS

FIG. 1 is a schematic illustration of thermally controlled isomerization of bisdibenzo-1,4-dioxocane between u-boat and s-boat conformers and how the incorporation of such contractile units into thermoset materials influences their thermal expansion and contraction behavior.

FIG. 2 is a schematic illustration of the molecular volume decrease during u-boat to s-boat isomerization of bisdibenzo-1,4-dioxocane.

FIG. 3 shows an exemplary synthetic route to a diamino-bisdibenzo-1,4-dioxocane.

FIG. 4 is an illustration of a method to synthesize polyamide modified with bisdibenzo-1,4-dioxocane.

FIG. 5 shows the reaction of a diamino-bisdibenzo-1,4-dioxocane with an epoxy resin to form an epoxy thermoset.

FIG. 6 shows the reaction of a dihydroxy-bisdibenzo-1,4-dioxocane with an isocyanate to form a polyurethane thermoset.

FIG. 7 shows the modeling results of 2,2′-diamino-bisdibenzo-1,4-dioxocane and 2,3′-diamino-bisdibenzo-1,4-dioxocane molecules in an epoxy resin to predict their thermodynamic and volume changes at 25° C. and 200° C.

FIG. 8 is a graph showing the glass transition temperatures of polyamide synthesized without a thermally contractile molecule, with dibenzocyclooctane, or with bisdibenzo-1,4-dioxocane.

FIG. 9 shows the change in length over temperature for the same series of polyamides in FIG. 8.

### DETAILED DESCRIPTION

Various technologies pertaining to polymer compositions having thermally contractile molecules and related methods are now described with reference to the drawings, wherein like reference numerals are used to refer to like elements throughout. In the following description, for purposes of explanation, numerous specific details are set forth in order to provide a thorough understanding of one or more aspects. It may be evident, however, that such aspect(s) may be practiced without these specific details.

### Definitions and Abbreviations

To further facilitate an understanding of the present disclosure, a number of terms and phrases are defined below. Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Ranges provided herein are understood to be shorthand for all of the values within the range. For example, a range of 1 to 25 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, or 25, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 25 may comprise 1 to 5, 1 to 10, 1 to 15, and 1 to 20 in one direction, or 25 to 20, 25 to 15, 25 to 10, and 25 to 5 in the other direction.

The term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

Additionally, as used herein, the term “exemplary” is intended to mean serving as an illustration or example of something, and is not intended to indicate a preference.

The abbreviation “CTE” as used herein is an abbreviation for the term “coefficient of thermal expansion.” The coefficient of thermal expansion is a measure of how the size of an object changes with a change in temperature. It is calculated by the fractional change in size per degree change in temperature, typically at a constant pressure, such that a lower CTE represents a material with a lower propensity for change in size upon change in temperature. CTEs may be determined for volumetric, area, and/or linear changes in size of a given object.

The abbreviation “DA-DBCO” as used herein is an abbreviation for the term “diamino-dibenzocyclooctane” and derivatives thereof. The molecular structure of DA-DBCO is displayed below.

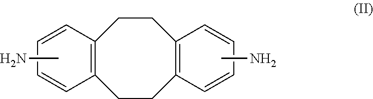

### Polymer Compositions

In general, a polymer composition according to the present disclosure comprises a polymeric moiety covalently bound to a bisdibenzo-1,4-dioxocane. As will be described hereinbelow, a substituted-bisdibenzo-1,4-dioxocane may be a thermally contractile molecule. Accordingly, the bisdibenzo-1,4-dioxocane is effective to reduce the CTE of the polymer composition as compared to the CTE of the polymer moiety while enabling the polymer moiety to maintain its properties, such as the modulus, the glass transition temperature (T_{g}), and the like.

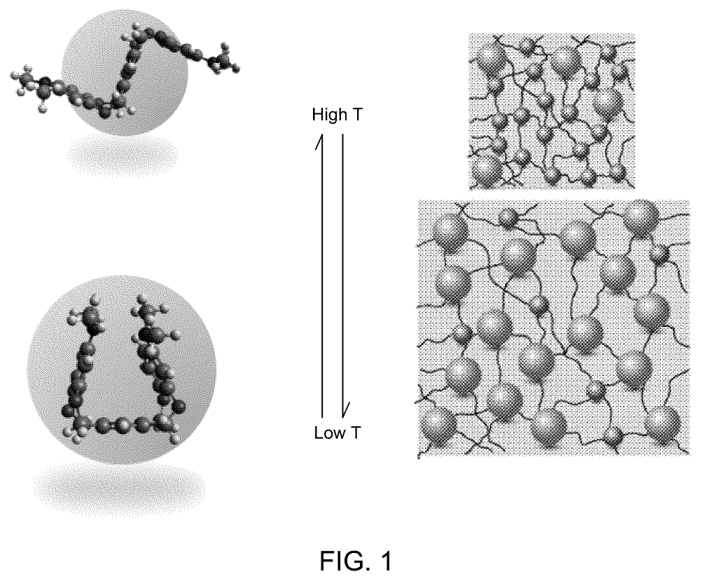

A strategy to manipulate the CTE of thermosets and/or thermoplastics involves covalent incorporation of a thermally contractile bisdibenzo-1,4-dioxocane within a polymer network, as illustrated in FIG. 1. These “shrinking” linkages oppose thermal expansion during heating, with the net effect of reducing the CTE of the material to near or less than zero in some cases. As shown in FIG. 1, one such contractile group is bisdibenzo-1,4-dioxocane, comprising two flexible cyclooctene rings with two oxygens each, interspersed between three rigid benzene rings, thereby providing high conformational flexibility. The cyclooctene rings undergo a reversible u-boat to s-boat isomerization upon heating accompanied by a decrease in molecular volume.

The polymer composition of various aspects comprises a polymeric moiety covalently bound to a bisdibenzo-1,4-dioxocane having the below structure:

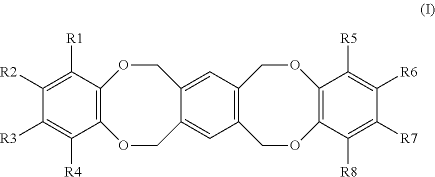

wherein at least one of R1-R8 comprises at least one covalent attachment site for the polymeric moiety; wherein the polymeric moiety is a polymer or a polymer resin; and wherein the polymer composition has a coefficient of thermal expansion (CTE) that is less than a coefficient of thermal expansion of the polymeric moiety.

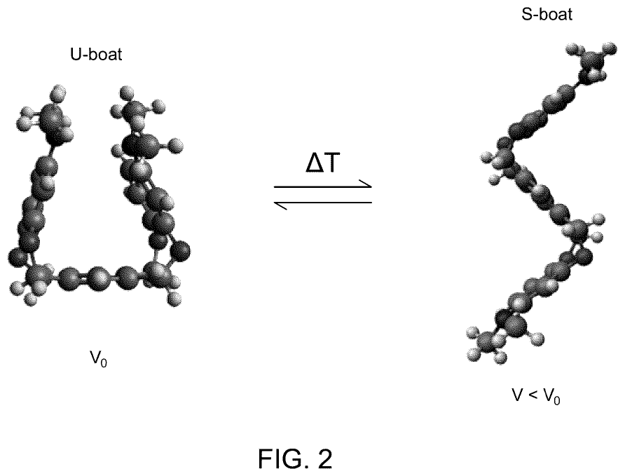

FIG. 2 shows the u-boat to s-boat transition for an exemplary bisdibenzo-1,4-dioxocane molecule, in which the molecular volume decreases upon this isomerization when heat is added to the system.

When the polymeric moiety is a polymer, the polymer composition may comprise a thermoplastic polymer composition. When the polymeric moiety is a polymer resin, the polymer composition may comprise a thermoset polymer composition. The polymer resin may be covalently bound to the at least one covalent attachment site by crosslinking. In aspects, the polymer moiety is selected from the group consisting of a polystyrene, a polyolefin, a polyurethane, a polyester, a polyamide, a polyimide, and a polyepoxide.

In aspects, the bisdibenzo-1,4-dioxocane comprises two or more covalent attachment sites for the polymeric moiety, with one or more covalent attachment sites at one or more of R1-R4 and one or more covalent attachment sites at one or more of R5-R8. In particular aspects, the bisdibenzo-1,4-dioxocane is a disubstituted-bisdibenzo-1,4-dioxocane having two covalent attachment sites, with one covalent attachment site at one of R1-R4 and another covalent attachment site at one of R5-R8.

In aspects, the disubstituted-bisdibenzo-1,4-dioxocane comprises a cis isomer, such as: a 1,1′-disubstituted-bisdibenzo-1,4-dioxocane; a 2,2′-disubstituted-bisdibenzo-1,4-dioxocane; a 3,3′-disubstituted-bisdibenzo-1,4-dioxocane; a 4,4′-disubstituted-bisdibenzo-1,4-dioxocane; a 1,2′-disubstituted-bisdibenzo-1,4-dioxocane; a 2,1′-disubstituted-bisdibenzo-1,4-dioxocane; a 3,4′-disubstituted-bisdibenzo-1,4-dioxocane; or a 4,3′-disubstituted-bisdibenzo-1,4-dioxocane. Alternatively, the disubstituted-bisdibenzo-1,4-dioxocane comprises a trans isomer, such as: a 1,3′-disubstituted-bisdibenzo-1,4-dioxocane; a 1,4′-disubstituted-bisdibenzo-1,4-dioxocane; a 2,3′-disubstituted-bisdibenzo-1,4-dioxocane; a 2,4′-disubstituted-bisdibenzo-1,4-dioxocane; a 3,1′-disubstituted-bisdibenzo-1,4-dioxocane; a 3,2′-disubstituted-bisdibenzo-1,4-dioxocane; a 4, 1′-disubstituted-bisdibenzo-1,4-dioxocane; or a 4,2′-disubstituted-bisdibenzo-1,4-dioxocane.

In aspects, each of the at least one covalent attachment sites comprise a first covalent bond between a functional group substituent and the bisdibenzo-1,4-dioxocane, and a second covalent bond between the functional group substituent and the polymeric moiety. In aspects, the functional group substituent on the bisdibenzo-1,4-dioxocane is selected from the group consisting of: an amine, an epoxide, an azide, a hydroxy, an isocyanate, a carboxylic acid, a vinyl, an acrylate, a methylacrylate, and an anhydride group.

The molecular structure of an exemplary polymer composition comprising a polyamide-containing 2,2′-bisdibenzo-1,4-dioxocane molecule is shown below.

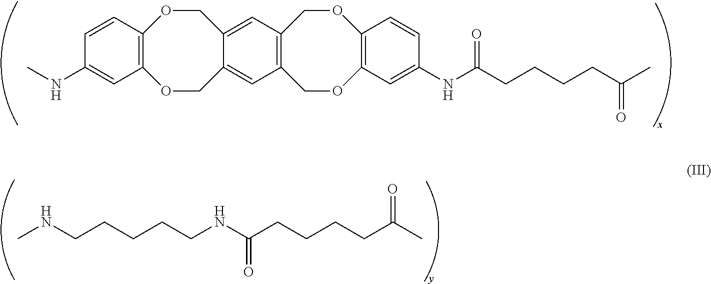

In aspects, x=0.1 and y=0.9.

The phenyl rings of a polymer moiety-substituted bisdibenzo-1,4-dioxocane can be further substituted with one or more alkyl groups such that the molecule can still undergo reversible u-boat to s-boat isomerization. Additionally, one or more of the “open” carbons in the cyclooctene rings (i.e., the carbons that are not also a part of the benzene rings) may have a side group substituent other than hydrogen. For example, the side group substituent(s) may comprise: a primary amine (NH₂), an alcohol (OH), a secondary amide (NH(CO)R′), or a ketone (C═O); or anyone of these groups attached to a short (C1-C3)alkane.

### Methods of Synthesizing a Substituted Bisdibenzo-1,4-Dioxocane

In general, a method for synthesizing a substituted bisdibenzo-1,4-dioxocane comprises adding a functional group reactant to a bisdibenzo-1,4-dioxocane intermediate to form a substituted bisdibenzo-1,4-dioxocane of structure (I).

More particularly, a method for synthesizing a substituted bisdibenzo-1,4-dioxocane comprises: (1) producing a reaction mixture comprising a bisdibenzo-1,4-dioxocane intermediate having at least one reactive moiety on at least one of its terminal benzene groups; (2) adding a functional group reactant to the reaction mixture; and (3) converting the at least one reactive moiety on the bisdibenzo-1,4-dioxocane intermediate to at least one functional group substituent to produce a substituted bisdibenzo-1,4-dioxocane having the below structure:

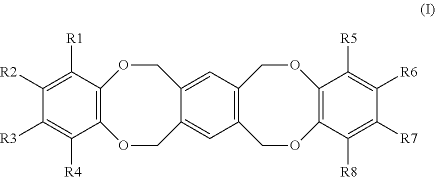

wherein at least one of R1-R8 comprises the at least one functional group substituent.

In aspects, there is more than one functional group substituent. When there is more than one functional group substituent, all of the functional group substituents may be the same. In particular aspects, the substituted bisdibenzo-1,4-dioxocane is a disubstituted bisdibenzo-1,4-dioxocane with two functional group substituents, wherein one of R1-R4 comprises the first functional group substituent, and one of R5-R8 comprises the second functional group substituent. The disubstituted bisdibenzo-1,4-dioxocane may be a cis isomer or a trans isomer.

In the method for synthesizing a substituted bisdibenzo-1,4-dioxocane, the addition step may further comprise adding a catalyst to the reaction mixture. For example, the catalyst can be selected from the group consisting of: a transition metal, a tri-tert-butylphosphine, a sodium nitrite, and a sodium-tert-butoxide. In aspects, the transition metal is selected from the group consisting of: palladium, platinum, rhodium, ruthenium, and iridium. In some particular aspects, the transition metal is palladium.

In the method for synthesizing a substituted bisdibenzo-1,4-dioxocane, the addition step may further comprise adding a stoichiometric reagent to the reaction mixture. For example, the stoichiometric reagent can be a superbase, such as an organolithium reagent. In some particular aspects, the stoichiometric reagent is BuLi.

In aspects, the at least one functional group substituent is selected from the group consisting of: an amine, an epoxide, an azido, a hydroxy, an isocyanate, a carboxylic acid, a vinyl, an acrylate, a methacrylate, and an anhydride group. In aspects, the functional group reactant is selected from the group consisting of: a metal-bis(trimethylsilyl)amide, an organic peroxide, a sodium azide, a bis(trimethylsilyl) peroxide, a phosgene, a bis(trichloromethyl) carbonate, a carbon dioxide, a tributylvinyltin, a methyl acrylate, a methyl methacrylate, and an acetyl chloride. In aspects, the at least one reactive moiety on the bisdibenzo-1,4-dioxocane intermediate is selected from the group consisting of: a halide, a vinyl, a diamine, a hydroxyl, and a carboxylic acid. In aspects, the halide is selected from the group consisting of: bromine, iodine, and chlorine.

In aspects, when the at least one functional group substituent is an amine, the functional group reactant is metal-bis(trimethylsilyl)amide, the at least one reactive moiety is a halide (e.g., bromine), and the catalyst comprises a transition metal (e.g., palladium) and a tri-tert-butylphosphine. In aspects, when the at least one functional group substituent is an epoxide, the functional group reactant is an organic peroxide and the at least one reactive moiety is a vinyl. In aspects, when the at least one functional group substituent is an azido, the functional group reactant is sodium azide, the at least one reactive moiety is a diamino compound, and the catalyst is sodium nitrite. In aspects, when the at least one functional group substituent is a hydroxy, the functional group reactant is a bis(trimethylsilyl) peroxide, the at least one reactive moiety is a halide (e.g., bromine), and THF and hydrochloric acid are added to the solution following the addition of the functional group reactant. In aspects, when the at least one functional group substituent is an isocyanate, the functional group reactant is a phosgene or a bis(trichloromethyl) carbonate and the at least one reactive moiety is a diamine. In aspects, when the at least one functional group substituent is a carboxylic acid, the functional group reactant is carbon dioxide, the at least one reactive moiety is a halide (e.g., bromine), and the stoichiometric reagent is BuLi. In aspects, when the at least one functional group substituent is a vinyl, the functional group reactant is tributylvinyltin, the at least one reactive moiety is a halide (e.g., bromine), and the catalyst is a transition metal (e.g., palladium). In aspects, when the at least one functional group substituent is an acrylate, the functional group reactant is a methyl acrylate (i.e., methacrylate), the at least one reactive moiety is a hydroxy, and the catalyst is a sodium tert-butoxide. In aspects, when the at least one functional group substituent is a methacrylate, the functional group reactant is a methyl methacrylate, the at least one reactive moiety is a hydroxy, and the catalyst is a sodium tert-butoxide. In aspects, when the at least one functional group substituent is an anhydride, the functional group reactant is an acetyl chloride, and the at least one reactive moiety is a carboxylic acid.

In aspects, when the at least one functional group substituent comprises an amine, a method for synthesizing a diamino-bisdibenzo-1,4-dioxocane comprises (1) mixing a 4-halocatechol and a 1,2,4,5-tetra(halomethyl)benzene with a potassium carbonate, an acetonitrile, and a dimethylformamide to produce at least one dihalide-bisdibenzo-1,4-dioxocane; and (2) adding a metal-bis(trimethylsilyl)amide, a transition metal, and a tri-tert-butylphosphine to the at least one dihalide-bisdibenzo-1,4-dioxocane, thereby converting the halide substituents of the at least one dihalide-bisdibenzo-1,4-dioxocane to functional group substituents via a Buchwald-Hartig amination to produce at least one disubstituted-bisdibenzo-1,4-dioxocane.

In aspects, the halide in the 4-halocatchol, the 1,2,4,5-tetra(halomethyl)benzene, and the dihalide-bisdibenzo-1,4-dioxocane is selected from the group consisting of: bromine, iodine, and chlorine. In some particular aspects, the halide is bromine.

When the dihalide-bisdibenzo-1,4-dioxocane is a cis isomer of a dihalide-bisdibenzo-1,4-dioxocane, the dihalide-bisdibenzo-1,4-dioxocane can be, for example, a 1,1′-dihalide-bisdibenzo-1,4-dioxocane; a 2,2′-dihalide-bisdibenzo-1,4-dioxocane; a 3,3′-dihalide-bisdibenzo-1,4-dioxocane; a 4,4′-dihalide-bisdibenzo-1,4-dioxocane; a 1,2′-dihalide-bisdibenzo-1,4-dioxocane; a 2, 1′-dihalide-bisdibenzo-1,4-dioxocane; a 3,4′-dihalide-bisdibenzo-1,4-dioxocane; or a 4,3′-dihalide-bisdibenzo-1,4-dioxocane. When the dihalide-bisdibenzo-1,4-dioxocane is a trans isomer of a dihalide-bisdibenzo-1,4-dioxocane, the dihalide-bisdibenzo-1,4-dioxocane can be, for example, a 1,3′-dihalide-bisdibenzo-1,4-dioxocane; a 1,4′-dihalide-bisdibenzo-1,4-dioxocane; a 2,3′-dihalide-bisdibenzo-1,4-dioxocane; a 2,4′-dihalide-bisdibenzo-1,4-dioxocane; a 3,1′-dihalide-bisdibenzo-1,4-dioxocane; a 3,2′-dihalide-bisdibenzo-1,4-dioxocane; a 4,1′-dihalide-bisdibenzo-1,4-dioxocane; or a 4,2′-dihalide-bisdibenzo-1,4-dioxocane.

The metal in the metal-bis(trimethylsilyl)amide used in the reaction can be selected from the group consisting of: lithium, sodium, and potassium. In some particular aspects, the metal-bis(trimethylsilyl)amide is a lithium bis(trimethylsilyl)amide.

The method may further include adding a dilute aqueous acid in a miscible organic solvent following the Buchwald-Hartig amination. The dilute aqueous acid and miscible organic solvent help to hydrolyze the trimethylsilane protecting groups from the amine in the metal-bis(trimethylsilyl)amide. In aspects, the dilute aqueous acid is hydrochloric acid. In aspects, the miscible organic solvent is tetrahydrofuran.

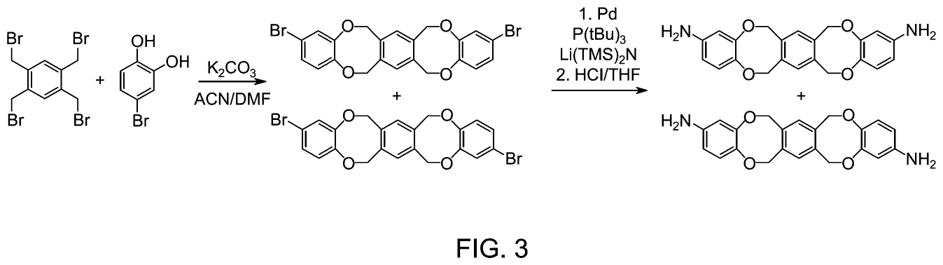

FIG. 3 shows an exemplary synthesis of 2,2′-diamino-bisdibenzo-1,4-dioxocane and 2,3′-diamino-bisdibenzo-1,4-dioxocane. In the first step, 4-bromocatechol and 1,2,4,5-tetra(bromomethyl)benzene are reacted with potassium carbonate, acetonitrile, and dimethylformamide to generate 2,2′-dibromo-bisdibenzo-1,4-dioxocane and 2,3′-dibromo-bisdibenzo-1,4-dioxocane. Next, the transitional metal palladium, Li (TMS) 2N, and potassium tert-butoxide are mixed with the dibromo-bisdibenzo-1,4-dioxocanes. Subsequently, hydrochloric acid and tetrahydrofuran are added to the reaction mixture to hydrolyze the trimethylsilane protecting groups from the amine. As a result of these final two reaction steps, 2,2′-diamino-bisdibenzo-1,4-dioxocane and 2,3′-diamino-bisdibenzo-1,4-dioxocane are formed.

### Methods of Preparing a Polymer Composition

In general, a method for preparing a polymer composition comprises (1) mixing a polymeric moiety with a substituted bisdibenzo-1,4-dioxocane having the structure (I) above with at least one functional group substituent; and (2) covalently bonding the polymeric moiety to the functional group substituent(s) of the disubstituted-bisdibenzo-1,4-dioxocane. The polymeric moiety is selected from the group consisting of: a monomer, a polymer, and a polymer resin. The polymer composition comprises the substituted bisdibenzo-1,4-dioxocane covalently bound to the polymer, the polymer resin, or the monomer which has subsequently undergone polymerization.

In the synthesis of the low CTE polymer composition, the polymeric moiety forms a covalent bond with the substituted bisdibenzo-1,4-dioxocane. Alternatively, the mixing step further includes mixing a linking group with the polymeric moiety and the substituted bisdibenzo-1,4-dioxocane, and then the linking group forming a first covalent bond with the polymeric moiety and a second covalent bond with the functional group substituent(s) of the substituted bisdibenzo-1,4-dioxocane.

When the polymeric moiety is a polymer or a polymer resin, the polymeric moiety can be selected from the group consisting of a polystyrene, a polyolefin, a polyurethane, a polyester, a polyamide, a polyimide, and a polyepoxide. Accordingly, the polymer composition can be selected from the group consisting of a polystyrene-bisdibenzo-1,4-dioxocane, a polyolefin-bisdibenzo-1,4-dioxocane, a polyurethane-bisdibenzo-1,4-dioxocane, a polyester-bisdibenzo-1,4-dioxocane, a polyamide-bisdibenzo-1,4-dioxocane, a polyimide-bisdibenzo-1,4-dioxocane, and a polyepoxide-bisdibenzo-1,4-dioxocane. When the polymeric moiety is a polymer, the polymer composition may be a thermoplastic polymer composition. When the polymeric moiety is a polymer resin, the polymer composition may be a thermoset resin.

When the polymeric moiety is a monomer, the method may further comprise a polymerization of the monomer following the covalent bonding step. In aspects, the monomer is selected from the group consisting of: a styrene, an olefin, a carbamate, an ester, an amide, an imide, and an epoxy. In aspects, the polymer composition comprises the substituted bisdibenzo-1,4-dioxocane covalently bound to the polymerized monomer.

In aspects, the functional group substituents are selected from the group consisting of: an amine, an epoxide, an azide, a hydroxy, an isocyanate, a carboxylic acid, a vinyl, an acrylate, a methacrylate, and an anhydride group.

In aspects, there is more than one functional group substituent. In particular aspects, the substituted bisdibenzo-1,4-dioxocane is a disubstituted bisdibenzo-1,4-dioxocane with two functional group substituents, wherein one of R1-R4 comprises the first functional group substituent, and one of R5-R8 comprises the second functional group substituent. When there is more than one functional group substituent, all of the functional group substituents may be the same. For example, the disubstituted-bisdibenzo-1,4-dioxocane can be diamino-bisdibenzo-1,4-dioxocane, diepoxide-bisdibenzo-1,4-dioxocane, diazido-bisdibenzo-1,4-dioxocane, dihydroxy-bisdibenzo-1,4-dioxocane, diisocyanate-bisdibenzo-1,4-dioxocane, dicarboxylic acid-substituted-bisdibenzo-1,4-dioxocane, divinyl-bisdibenzo-1,4-dioxocane, diacrylic acid-substituted-bisdibenzo-1,4-dioxocane, dimethylacrylic acid-substituted-bisdibenzo-1,4-dioxocane, or dianhydride-bisdibenzo-1,4-dioxocane.

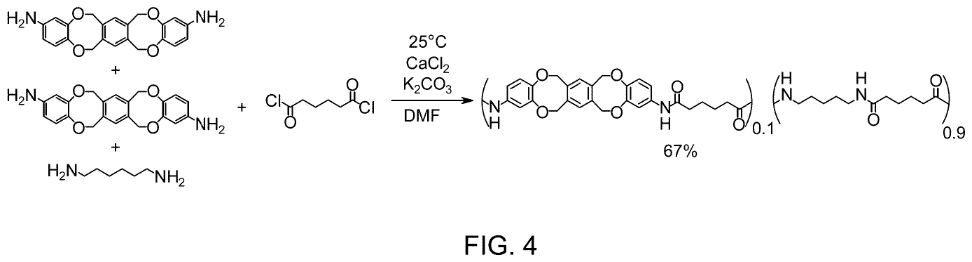

FIG. 4 is an illustration of a method to synthesize polyamide-containing bisdibenzo-1,4-dioxocane. This reaction involves combination of 2,2′-bisdiaminodibenzo-1,4-dioxocane, 2,3′-diamino-bisdibenzo-1,4-dioxocane, hexamethylenediamine, and adipoyl chloride with calcium chloride, potassium carbonate, and dimethylformamide at 150° C. This reaction produces a low CTE polyamide wherein the polymer comprises 10 wt. % of the bisdibenzo-1,4-dioxocane.

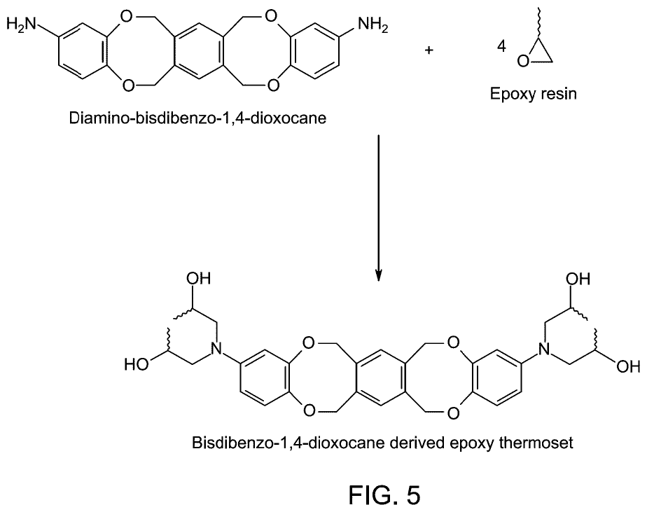

FIG. 5 is an illustration of a method to synthesize an epoxy containing bisdibenzo-1,4-dioxocane by cross linking an epoxy resin with a diamino-bisdibenzo-1,4-dioxocane.

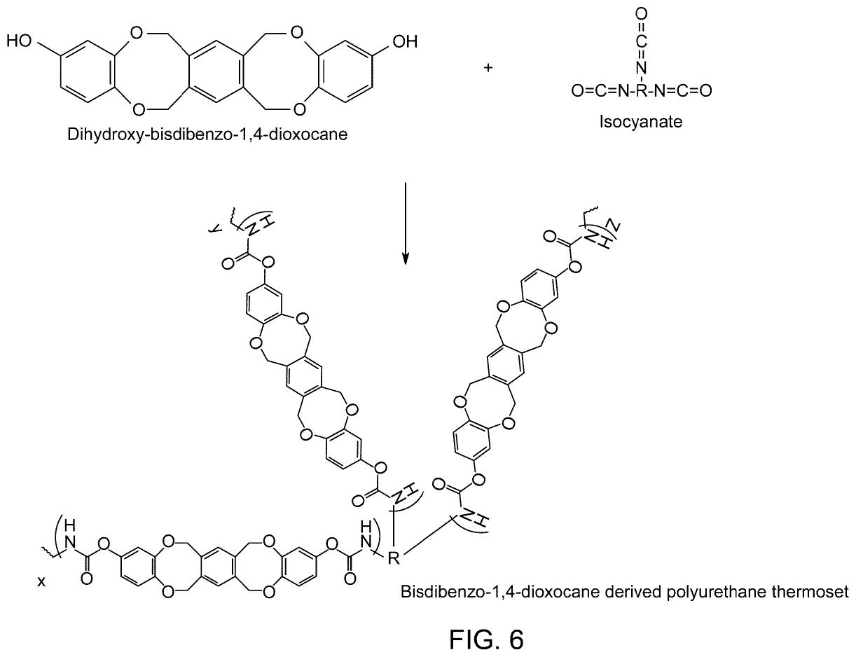

The synthetic approaches also lend themselves to the creation of dialcohol-bisdibenzo-1,4-dioxocanes or diisocyanate-bisdibenzo-1,4-dioxocanes, which can be incorporated into the backbones of polyurethanes. For example, an isocyanate can be crosslinked with dihydroxy-bisdibenzo-1,4-dioxocane, as shown in FIG. 6, or a polyol can be crosslinked with diisocyanate-bisdibenzo-1,4-dioxocane to provide a polyurethane-containing bisdibenzo-1,4-dioxocane.

Although any methods and materials similar or equivalent to those described herein can be used in the practice or testing of the present disclosure, the preferred methods and materials are described. Other features, objects, and advantages of the present disclosure will be apparent from the description and the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All references cited herein are incorporated herein by reference in their entirety and for all purposes to the same extent as if each individual publication, patent, or patent application was specifically and individually indicated to be incorporated by reference in its entirety for all purposes.

### Examples

The following examples are included for the purposes of illustration and does not limit the scope of the general inventive concepts described herein.

### Example 1: Modeling of Disubstituted-Bisdibenzo-1,4-Dioxocane Conformations at Low and High Temperatures

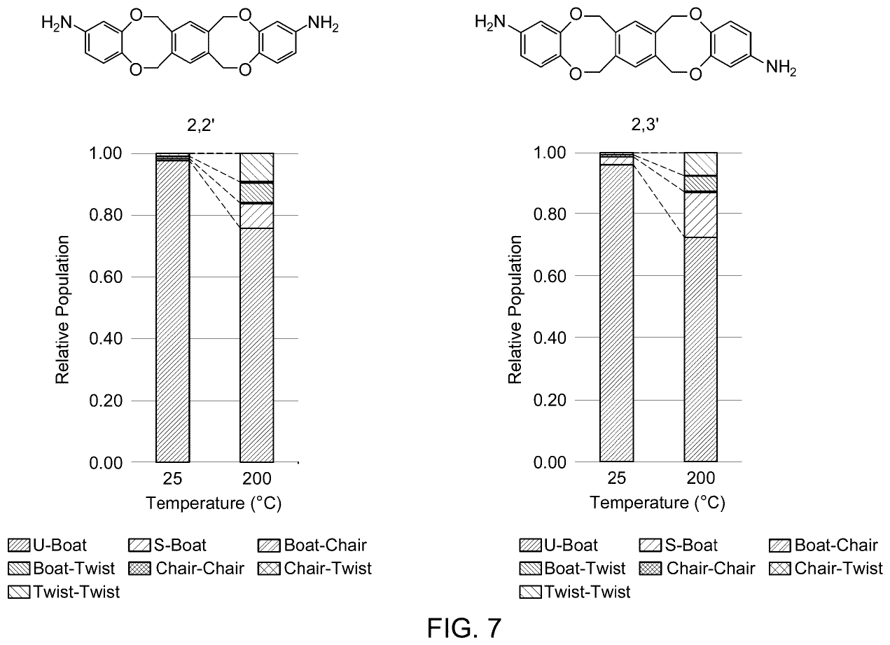

The conformational changes of disubstituted bisdibenzo-1,4-dioxocane were modeled in an epoxy resin to predict the thermodynamic and volume changes of these molecules at 25° C. and 200° C. The results are shown in FIG. 7. Both 2,2′-diamino-bisdibenzo-1,4-dioxocane and 2,3′-diamino-bisdibenzo-1,4-dioxocane were predicted to have a higher proportion of lower volume conformations at 200° C. than at the lower temperature.

### Example 2: Transition Temperature of Polyamide Synthesized with Low CTE Molecules

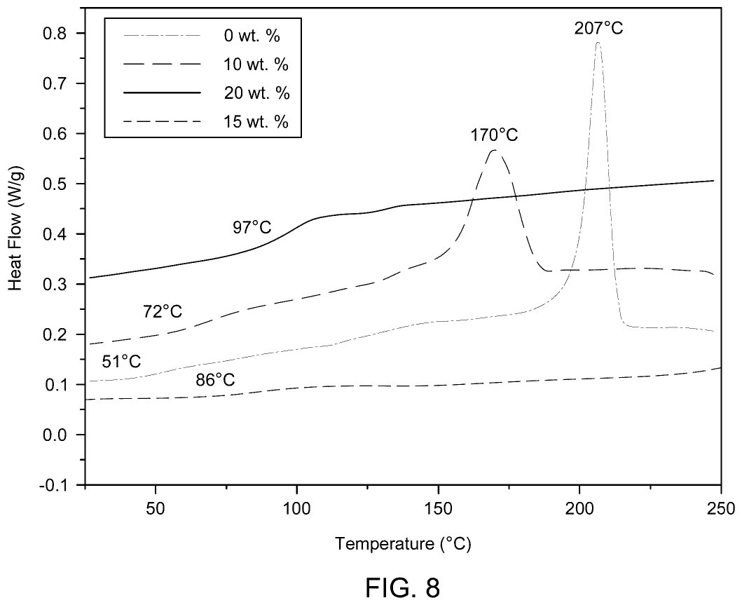

Polyamide-containing bisdibenzo-1,4-dioxocane was synthesized according to the above methods, as shown in FIG. 4. Bisdibenzo-1,4-dioxocane was included in the reaction mixture at the following weight percents: 0 wt. %, and 15 wt. %. As a comparison, polyamide was synthesized with dibenzocyclooctane (a known, thermally contractile molecule) at the following weight percents: 10 wt. % and 20 wt. %. The glass transition temperature of each of these compositions was determined, and the results are shown in FIG. 8 and Table 1 (below).

TABLE 1

| | Low CTE Molecule Wt. % | | | |
|---|---|---|---|---|
| | 0 | 10 (DBCO) | 15 (BDB-DO) | 20 (DBCO) |
| Transition Temp (° C.) | 51 | 72 | 86 | 97 |

As can be seen from the data, the polyamide-containing bisdibenzo-1,4-dioxocane had a similar glass transition temperature as the DBCO compositions, while at a lower heat flow.

### Example 3: Change in Length of Polymers Compositions Containing Low CTE Molecules

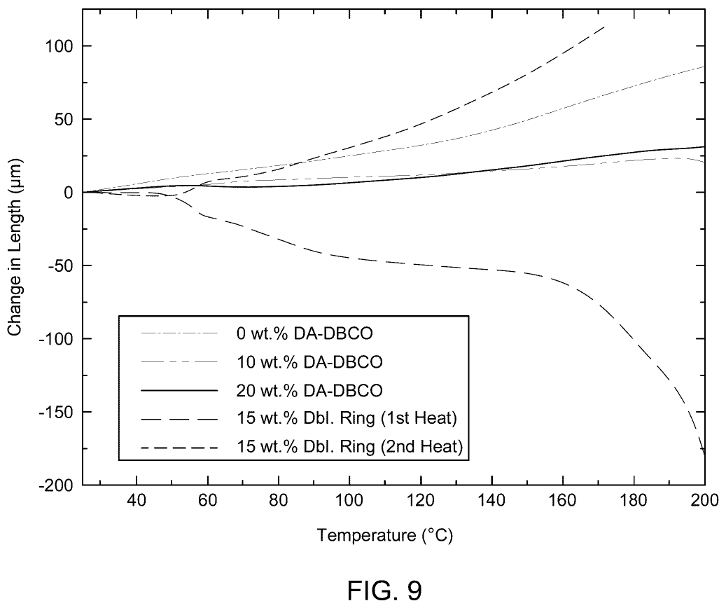

Polymers compositions containing DA-DBCO and bisdibenzo-1,4-dioxocane were synthesized, with the low CTE molecules being added at varying percents to the polymer resins. The change in length of these modified polymers were then measured over a change in temperature from 20° C. to 200° C. All DA-DBCO polyamide data is from the second heating cycle. The results are shown in FIG. 9, with bisdibenzo-1,4-dioxocane shown in the key as “Dbl. Ring.” The polyamide containing bisdibenzo-1,4-dioxocane demonstrated a substantial shrinking upon an increase in temperature upon the first heating. The second heating of the polyamide containing bisdibenzo-1,4-dioxocane exhibited an expansion upon heating, although the second heating was done quite quickly following the first heating (i.e., in the range of a few minutes). It is expected that, if the sample is allowed to fully cool between heating cycles, a shrinking upon increase in temperature similar to the first heating will be observed.

What has been described above includes examples of one or more aspects. It is, of course, not possible to describe every conceivable modification and alteration of the above compositions or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A polymer composition comprising:
a polymeric moiety covalently bound to a bisdibenzo-1,4-dioxocane having the below structure: 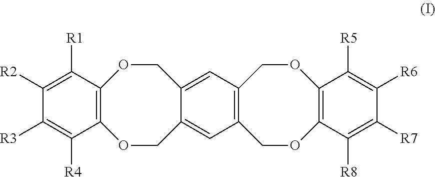
wherein at least one of R1-R8 comprises at least one covalent attachment site for the polymeric moiety;
wherein the polymeric moiety is a polymer or a polymer resin; and
wherein the polymer composition has a coefficient of thermal expansion (CTE) that is less than a coefficient of thermal expansion of the polymeric moiety.

2. The polymer composition of claim 1, wherein the at least one covalent attachment site comprises a first covalent bond between a functional group substituent and the bisdibenzo-1,4-dioxocane, and a second covalent bond between the functional group substituent and the polymeric moiety.

3. The polymer composition of claim 1, wherein the polymeric moiety is selected from the group consisting of: a polystyrene, a polyolefin, a polyurethane, a polyester, a polyamide, a polyimide, and a polyepoxide.

4. The polymer composition of claim 1, wherein thebisdibenzo-1,4-dioxocane comprises two or more covalent attachment sites, with one or more covalent attachment sites at one or more of R1-R4 and one or more covalent attachment sites at one or more of R5-R8.

5. The polymer composition of claim 1, wherein thebisdibenzo-1,4-dioxocane is a disubstituted-bisdibenzo-1,4-dioxocane having two covalent attachment sites, with one covalent attachment site at one of R1-R4 and another covalent attachment site at one of R5-R8.

6. A method for synthesizing a substituted-bisdibenzo-1,4-dioxocane, the method comprising:
producing a reaction mixture comprising a bisdibenzo-1,4-dioxocane intermediate having at least one reactive moiety on at least one of its terminal benzene groups;
adding a functional group reactant to the reaction mixture; and
converting the at least one reactive moiety on the bisdibenzo-1,4-dioxocane intermediate to at least one functional group substituent to produce a substituted bisdibenzo-1,4-dioxocane having the below structure: 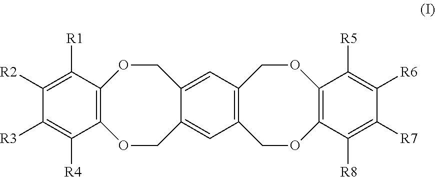
wherein at least one of R1-R8 comprises the at least one functional group substituent.

7. The method of claim 6, wherein the substituted bisdibenzo-1,4-dioxocane is a disubstituted bisdibenzo-1,4-dioxocane with two functional group substituents, wherein one of R1-R4 comprises the first functional group substituent, and one of R5-R8 comprises the second functional group substituent.

8. The method of claim 6, wherein there is more than one functional group substituent, and wherein all of the functional group substituents are the same.

9. The method of claim 6, wherein the at least one functional group substituent is selected from the group consisting of: an amine, an epoxide, an azido, a hydroxy, an isocyanate, a carboxylic acid, a vinyl, an acrylate, a methacrylate, and an anhydride group.

10. The method of claim 6, wherein the functional group reactant is selected from the group consisting of: a metal-bis(trimethylsilyl)amide, an organic peroxide, a sodium azide, a bis(trimethylsilyl) peroxide, a phosgene, a bis(trichloromethyl) carbonate, a carbon dioxide, a tributylvinyltin, a methyl acrylate, a methyl methacrylate, and an acetyl chloride.

11. The method of claim 6, wherein the at least one reactive moiety on the bisdibenzo-1,4-dioxocane intermediate is selected from the group consisting of: a halide, a vinyl, a diamine, a hydroxyl, and a carboxylic acid.

12. The method of claim 6, wherein the addition step further comprises adding a catalyst to the reaction mixture, and wherein the catalyst is selected from the group consisting of: a transition metal, a tri-tert-butylphosphine, a sodium nitrite, and a sodium-tert-butoxide.

13. The method of claim 12, wherein:
the step of producing the reaction mixture comprises mixing a 4-halocatechol and a 1,2,4,5-tetra(halomethyl)benzene with a potassium carbonate, an acetonitrile, and a dimethylformamide;
the at least one reactive moiety comprises two halides and the bisdibenzo-1,4-dioxocane intermediate comprises a dihalide-bisdibenzo-1,4-dioxocane having one halide on each of its terminal benzene groups;
the functional group reactant comprises a metal-bis(trimethylsilyl)amide;
the catalyst comprises a transition metal and a tri-tert-butylphosphine; and
the at least one functional group substituent comprises two amines, wherein one of R1-R4 comprises the first amine, and one of R5-R8 comprises the second amine.

14. The method of claim 13, wherein the metal in the metal-bis(trimethylsilyl)amide is selected from the group consisting of: lithium, sodium, and potassium.

15. The method of claim 13, wherein the method further comprises adding a dilute aqueous acid in a miscible organic solvent to the reaction mixture following the addition of the functional group reactant and the catalyst.

16. A method of preparing a polymer composition, the method comprising:
mixing a polymeric moiety with a substituted-bisdibenzo-1,4-dioxocane having the below structure: 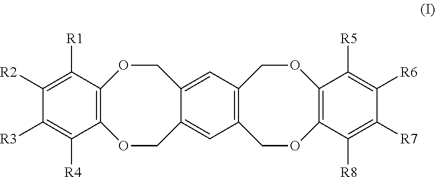
wherein at least one of R1-R8 comprises at least one functional group substituent and wherein the polymeric moiety is selected from the group consisting of: a monomer, a polymer, and a polymer resin; and
covalently bonding the at least one functional group substituent of the substituted bisdibenzo-1,4-dioxocane to the polymeric moiety.

17. The method of claim 16, wherein the substituted bisdibenzo-1,4-dioxocane is a disubstituted bisdibenzo-1,4-dioxocane with two functional group substituents, wherein one of R1-R4 comprises the first functional group substituent, and one of R5-R8 comprises the second functional group substituent.

18. The method of claim 16, wherein the polymeric moiety is a polymer or polymer resin, and wherein the polymeric moiety is selected from the group consisting of: a polystyrene, polyolefin, a polyurethane, a polyester, a polyamide, a polyimide, and an epoxy.

19. The method of claim 16, wherein the mixing step further comprises mixing a linking group with the polymeric moiety with and the substituted-bisdibenzo-1,4-dioxocane; and
wherein the covalent bonding step comprises the linking group forming a covalent bond with the polymeric moiety, and the linking group forming a covalent bond with the at least one functional group substituent.

20. The method of claim 16, wherein the polymeric moiety is a monomer selected from the group consisting of: a styrene, an olefin, a carbamate, an ester, an amide, an imide, and an epoxy; and
wherein the method further comprises a polymerization of the monomer following the covalent bonding step.
